(19) United States
(12) Patent Application Publication
(10) Pub. No.: US 2023/0285483 A1
(43) Pub. Date: Sep. 14, 2023

(54) TINCTURE CONTAINING CANNABINOIDS, METHOD OF USING, AND METHOD OF MAKING THE SAME

(71) Applicant: Holistic Hemp Solutions, Inc., Norton, MA (US)

(72) Inventors: Julie A. Mejia, Norton, MA (US); Stephen Hunt, Casa Grande, AZ (US)

(21) Appl. No.: 18/179,863

(22) Filed: Mar. 7, 2023

Related U.S. Application Data
(60) Provisional application No. 63/317,717, filed on Mar. 8, 2022.

(51) Int. Cl. A61K 36/185; A61K 31/05; A61K 31/352; A61K 31/015; A61K 31/045; A61K 31/01; A61K 47/44; A61K 31/60

(52) U.S. Cl. CPC A61K 36/185; A61K 31/05; A61K 31/352; A61K 31/015; A61K 31/045; A61K 31/01; A61K 47/44; A61K 31/60; A61K 2236/35

(57) ABSTRACT

A tincture is disclosed that contains substantially all of the cannabinoids and terpenes extracted from processed hemp. Methods of using the tincture are disclosed including preventing or treating a coronavirus, reducing or treating arthritic pain or muscle pain, and reducing or treating anxiety. Also disclosed is a method of making the tincture by cold extraction which allows extraction of substantially all of the terpenes and cannabinoids from the hemp.

### CROSS-REFERENCE TO RELATED APPLICATION

The present disclosure claims the benefit of priority of U.S. Provisional Application No. 63/317,717, filed on Mar. 8, 2022, the entire contents of which are incorporated by reference herein.

### FIELD OF THE DISCLOSURE

The disclosure relates to a tincture containing cannabinoids and terpenes extracted from hemp, and a carrier oil, prepared by a cold processing methodology. The tincture may be used to prevent or treat arthritis, muscle aches or pains, seizure disorders, anxiety, depression, coronavirus, and/or other diseases and conditions.

### BACKGROUND OF THE DISCLOSURE

Traditional edible and tincture products made from hemp use heat extraction, which destroys many of the beneficial terpenes and cannabinoids. As such, the final product does not contain the full scope of the terpenes and cannabinoids that were in the original hemp before heating and extraction.

When cannabis undergoes a heating process, the cannabinoid acids' molecular structure changes. When decarboxylated, the acids lose one carboxyl group (—COOH) as carbon dioxide while retaining one hydrogen atom. When a carboxyl group is removed, the molecular mass of THCA, for instance, decreases by about 12 percent turning into the active THC compound.

It is generally understood that decarboxylation is one of the most important processes when making edibles, tinctures, and other consumable goods, because there is no heat added during consumption of these products. Decarboxylation activates some of the plant's most essential cannabinoids, e.g., tetrahydrocannabinol (THC) and cannabidiol (CBD). Freshly harvested and un-decarboxylated cannabis flower contains an excess of cannabinoid acids that have little to no psychoactive benefit until processors decarboxylate them. The decarboxylation reaction achieved through heating converts tetrahydrocannabinolic acid (THCa) into THC, and cannabidiolic acid (CBDa) into CBD.

CBDa has been shown to treat or prevent pain, inflammation, anxiety, nausea, and seizures. CBGa is also known to have analgesic, antibacterial, anti-inflammatory, and anti-proliferative properties.

A study conducted by researchers at Oregon State University found that two compounds commonly found in hemp—cannabigerolic acid, or CBGa, and cannabidiolic acid, or CBDa—have potential to combat coronavirus. In the study, these compounds were found to bind to spike proteins found on the virus and blocked a step the pathogen uses to infect people. If the virus was already present, these compounds reduced severe symptomology.

In another study published in the Journal of Nature Products, cannabis compounds prevented the virus that causes Covid-19 from penetrating healthy human cells. (R. B. van Breeman, et al., “Cannabinoids Block Cellular Entry of SARS-CoV-2 and the Emerging Variants,” J Nat Prod. 2022 Jan. 28; 85(1):176-184.

There is a need for a product providing a user with an all-natural full spectrum terpene and cannabinoid blend that assists with a variety of different conditions.

### SUMMARY OF THE DISCLOSURE

A tincture is disclosed containing substantially all of the cannabinoids and terpenes extracted from hemp. The tincture is comprised of a carrier, such as a carrier oil. The tincture may include cannabinoids selected from the group consisting of: CBD, CBDa, CBDva, CBG, CBGa, CBC, CBCa, and any combination thereof, and optionally an additive, optionally for flavoring. The terpenes present in the tincture may include Beta-Myrcene, Beta-caryophyllene, Alpha-Pinene, Beta-Pinene, Camphene, Delta-3-carene, Alpha-Humulene, Alpha-Ocimene, and optionally one or more additional terpenes. The tincture may comprises about 0.01 wt. % to about 0.02 wt. % of Beta-Myrcene, about 0.01 wt. % to about 0.02 wt. % of Beta-caryophyllene, about 0.01 wt. % to about 0.02 wt. % of Alpha-Pinene, about 0.01 wt. % to about 0.02 wt. % of Beta-Pinene, about 0.01 wt. % to about 0.02 wt. % of Camphene, about 0.01 wt. % to about 0.02 wt. % of Delta-3-carene, and about 0.01 wt. % to about 0.02 wt. % of Alpha-Humulene.

The cannabinoids may be present in about 0.4 wt. % to about 1.5 wt. % by total weight of the tincture, and/or the terpenes may be present in about 0.1 wt. % to about 0.3 wt. %, by total weight of the tincture. The tincture may include: CBD in about 0.1 wt % to about 0.3 wt %; CBDa in about 0.1 wt % to about 0.3 wt %; CBGa in about 0.1 wt % to about 0.35 wt %; CBC in about 0.01 wt % to about 0.02 wt %; and CBCa in about 0.01 wt % to about 0.02 wt %, by total weight of the tincture.

Methods of using the tincture are disclosed including preventing or treating a coronavirus, reducing or treating arthritic pain or muscle pain, reducing or treating anxiety, reducing or treating inflammation, slowing tumor growth, reducing or treating symptoms of nausea and vomiting, stimulating appetite, and/or reducing seizure activity, by administering the tincture.

A method of making the tincture by cold extraction is disclosed. The method includes the steps of: i) supplying raw hemp; ii) placing the hemp in a light-blocking vessel; iii) adding a carrier to the vessel make a filled vessel; iv) mixing the hemp and carrier in the filled vessel to evenly distribute the ingredients; v) storing the filled vessel in a controlled environment for a period of time; and vi) mixing the filled vessel at least one time per week throughout the period of time.

### DETAILED DESCRIPTION OF THE DISCLOSURE

The tincture disclosed herein contains a combination of cannabinoids extracted from hemp and a carrier. The tincture is prepared using a cold extraction process which provides for extraction of substantially all of the terpenes and cannabinoids from the hemp material. Extraction is achieved without heat or press. The tincture contains substantially all, or all, of the unaltered cannabinoids extracted from the raw hemp.

The cold process extraction is applied to raw hemp that may be dried flower or fresh, recently harvested, e.g., with the prior 72 hours. The hemp may be selected from the following strains: Hawaiian Haze, Sour Space Candy, Super Sour Space Candy, Cherry Pie, Special Sauce, Lifter Plus, Elektra, Cherry Creme Brulee, Harlequin, Gorilla Glue (Hemp version), Hulk, Chardonnay, Cherry Wine, Bubba Kush (Hemp version), Haute Wife, Cheese (Hemp version), Santa Clara Haze, Gun Powder OG (Hemp version), Silver Haze (CBG Hemp version), Cherry #4, T-1 (Hemp version), White (CBG Hemp version), Sour G (CBG Hemp version), White Widow (CBG Hemp version), Jack Frost (Hemp version), Dessert Snow (CBG Hemp version), Lemon Octane, Blue Dream (Hemp version), Spec Diesel, Blueberry Muffin, Strawberry cake, Cherry Abcus, Magic Bullet, Pink Panther, Maui Wowi, Golden Kush, Red Bordeaux, Frosted Lime, Boaox, Bubblegum (Hemp version), Merlot, Super Lemon Haze (Hemp version), John Snow (Hemp version), Superglue (Hemp version), Lemon Cream Diesel, Stem Cell, White Whale, Frosted Cake, Stardust, Plain Jane Secrets Nature, Shiatsu, Matterhorn, Forbidden Fruit, Forbidden V, Zkittles (Hemp version), Bubba Cryo, Runtz (Hemp version), Pine Walker, and any combination thereof. The selected strains contain less than 0.3% Δ9-THC.

The cannabinoids present in the tincture and extracted from hemp may be selected from the group consisting of: CBD, CBDa, CBDva, CBG, CBGa, THCa, THCv, CBGv, CBGva, CBDv, CBCv, CBC, CBCa, CBGM, CBN, CBL, CBLa, and any combination thereof. The cannabinoids present in the tincture may be selected from the group consisting of: CBD, CBDa, CBDva, CBG, CBGa, CBC, CBCa, and any combination thereof. The cannabinoids present in the tincture and extracted from hemp may include CBD, CBDa, CBDva, CBG, CBGa, CBC, and CBCa.

The tincture may also include one or more terpenes extracted from hemp. The terpenes may be present in about 0.1 wt. % to about 0.3 wt. %, depending on the hemp used to make the tincture. The terpenes may be selected from the group consisting of: Myrcene, Beta-caryophyllene, Limonene, Linalool, Pinene, Humulene, Terpinolene, Alpha-bisabolol, Eucalyptol, Geraniol, Terpineol, Farnesene, Borneol, Ocimene, Nerolidol, Guaiol, Valencene, Delta-3-carene, Phytol, Sabinene, Phellandrene, Menthol, Fenchol, Terpinene, Isoborneol, Cymene, Octanol, Cedrene, Camphene, Geranyl Acetate, Bergamotene, Camphor, Pulegon, and any combination thereof. The tincture may include at least one or more of the following terpenes: Beta-Myrcene, Beta-caryophyllene, Alpha-Pinene, Beta-Pinene, Camphene, Delta-3-carene, Alpha-Humulene, Alpha-Ocimene. Where not specified, the forgoing may be alpha and/or beta. The tincture may include five or more different terpenes extracted from hemp, with at least five of the terpenes being present in about 0.01 wt. % to about 0.03 wt. %. The tincture may include eight or more different terpenes extracted from hemp, with at least six of the terpenes being present in about 0.01 wt. % to about 0.03 wt. %, or with at least six of the terpenes being present in about 0.02 wt. %.

The tincture may include at least a combination of the following terpenes: Beta-Myrcene, Beta-caryophyllene, Alpha-Pinene, Beta-Pinene, Camphene, Delta-3-carene, Alpha-Humulene, Alpha-Ocimene, optionally one or more additional terpenes, and optionally with each of Beta-Myrcene, Beta-caryophyllene, Alpha-Pinene, Beta-Pinene, Camphene, Delta-3-carene, Alpha-Humulene, and Alpha-Ocimene being present in about 0.01 wt. % to about 0.02 wt. %. The tincture may include Beta-Myrcene in about 0.02 wt. %, and Beta-caryophyllene in about 0.02 wt. %, and optionally, one or more additional terpenes in about 0.01 wt. %, and optionally one or more additional terpenes in about 0.02 wt. %, by total weight of the tincture.

The term “substantially all” as used herein in connection with the terpenes and/or cannabinoids means that over 90% of the terpenes and/or cannabinoids, as applicable, present in the raw hemp are extracted into the carrier and present in the tincture. This term applies to the individual type of terpene and cannabinoid not to the concentration of each one, which may vary after extraction. When “all” of the terpenes and cannabinoids are extracted from the hemp, this is understood to mean that each of the individual type of terpene and cannabinoid that was present in the hemp prior to processing is present in some amount in the tincture.

The carrier may be selected based on user preference and which oil delivers the best effect/metabolizes best for their endocannabic system and their body. The carrier may be all natural or naturally derived oil, all natural or naturally derived butter, all natural or naturally derived wax, or any combination thereof. The carrier may be selected from the group consisting of: Olive oil, Hempseed oil, Fractionated MCT/Coconut oil, Avocado oil, Epsom Salts, Magnesium (salt, oil and flakes), Bee and Carnauba Waxes, Coco Butter, Mango Butter, Shea Butter (raw yellow, refined white), Shea oil, Jojoba, Cod Liver oil, Sunflower oil, Peanut oil, any known vegetable oil, such as but not limited to canola or safflower, Almond butter, Almond oil, Glycerin (in all forms), Liquid Castile Soap, Emu and other animal derived oils, Vitamin E oil, Caster Oil, Zinc Oxide, Citric Acid, and other all natural essential oils and emulsifiers. The carrier may be a carrier oil selected from the group consisting of: Olive oil, Hempseed oil, Fractionated MCT/Coconut oil, Avocado oil, Almond oil, Cod Liver oil, Sunflower oil, Peanut oil, any known vegetable oil, such as but not limited to canola or safflower, or any combination thereof. The carrier may be an olive oil blend containing olive oil and one or more additional carriers. The carrier may be olive oil.

The tincture may contain one or more additives. The additive may be 100% natural. The additive may be a natural flavor, natural coloring agent, or natural preservative. The additive may be natural mint or spearmint. When present, the tincture may contain about 1 wt. % to about 20 wt. %, about 5 wt. % to about 20 wt. %, about 10 wt. % to about 20 wt. % of one or more additives. The tincture may be free of artificial additives, including but not limited to artificial flavors, colors, preservatives, and thickeners.

The tincture may contain about 98.0 wt % to about 99.9 wt % carrier, about 98.5 wt % to about 99.5 wt % carrier, about 99.0 wt % to about 99.4 wt % carrier, or about 99.1 wt % to about 99.3 wt % carrier. The tincture may contain about 0.4 wt % to about 1.5 wt % total cannabinoids, about 0.5 wt % to about 1.0 wt % total cannabinoids, about 0.6 wt % to about 0.9 wt % total cannabinoids, or about 0.6 wt % to about 0.8 wt % total cannabinoids. The tincture may contain about 0.1 wt % to about 0.4 wt %, or about 0.1 wt % to about 0.3 wt % CBD. The tincture may contain about 0.05 wt % to about 0.4 wt %, or about 0.1 wt % to about 0.3 wt % CBDa. The tincture may contain about 0.001 wt % to about 0.05 wt %, or about 0.002 wt % to about 0.006 wt % CBDva. The tincture may contain about 0.001 wt % to about 0.1 wt %, or about 0.01 wt % to about 0.05 wt % CBG. The tincture may contain about 0.05 wt % to about 0.4 wt %, or about 0.1 wt % to about 0.35 wt % CBGa. The tincture may contain about 0.01 wt % to about 0.1 wt %, about 0.01 wt % to about 0.04 wt %, or about 0.01 wt % to about 0.02 wt % CBC. The tincture may contain about 0.001 wt % to about 0.05 wt %, or about 0.01 wt % to about 0.02 wt % CBCa. The tincture may include about 0.1 wt % to about 0.3 wt % CBD, about 0.1 wt % to about 0.3 wt % CBDa, about 0.1 wt % to about 0.35 wt % CBGa, about 0.01 wt % to about 0.02 wt % CBC, about 0.01 wt % to about 0.02 wt % CBCa.

The tincture may be free of one or more of the following cannabinoids: Δ9-THC, CBN, CBNa, THCa, Δ8-THC, THCv, THCva, CBDv, CBL, CBLa, CBCv, and CBT. The tincture may be free of all of the following cannabinoids: Δ9-THC, CBN, CBNa, THCa, Δ8-THC, THCv, THCva, CBDv, CBL, CBLa, CBCv, and CBT. The tincture may contain less than 0.3 wt. % of 49-THC.

The tincture of the disclosure contains high amounts of CBDa (Cannabidiolic Acid), the precursor to CBD (Cannabidiol) that is converted via decarboxylation. This makes the tincture unique, as it may be consumed it in two ways. The tincture disclosed herein contains detectable amounts of CBDa (Cannabidiolic Acid) which may optionally be converted by the user with application of heat. Other products on the market do not contain CBDa.

CBDva (cannabidivarinic acid) has shown promising results for easing anxiety, slowing tumor growth, reducing symptoms of nausea and vomiting, stimulating appetite and reducing seizure activity. Research shows it helps reduce pain and inflammation differently than that of opioids. CBDva processes through the central nervous system by blocking pain signs to the brain unlike Opioids which attach to proteins called opioid receptors on nerve cells in the brain, spinal cord, gut, and other parts of the body. When this happens, the opioids block pain messages sent from the body through the spinal cord to the brain.

The tincture of the disclosure is made using a cold processing methodology that extracts substantially all, or all, of the cannabinoids and terpenes found in the raw hemp into the carrier. Thus, cold processing extraction maintains the cannabinoids and terpenes present in the raw hemp and is able to extract them into the carrier and into the tincture for superior results and breadth of cannabinoids and terpenes.

Cold extraction processing, also referred to as cold extraction, may take about 4 weeks to about 14 weeks, or about 4 weeks to about 8 weeks. The length of time may vary depending on the desired cannabinoid profile, temperature and pressure of the environment (e.g., season, and location of laboratory), and whether the raw hemp was grown from seeds and/or clones, and the growing methodology that was applied.

Cold extraction processing includes: supplying raw hemp; placing the hemp in a light-blocking vessel; adding a carrier, and optionally an additive, to the vessel; mixing the hemp and carrier in the filled vessel to evenly distribute the ingredients; storing the filled vessel in a controlled environment for a period of time; and mixing the filled vessel at least one time per week throughout the period of time.

Enough carrier may be added to sufficiently cover, or submerge, the hemp. When present, the carrier and additive may be mixed in a separate container of any kind before adding to the vessel contain the raw hemp. The carrier may be olive oil or an olive oil blend and, when present, the additive may be mint or spearmint leaf.

The filled vessel may be mixed, or otherwise agitated, either by hand and or by machine and may be mixed at a minimum of once weekly. It may be mixed once weekly, twice weekly, three times weekly or more often. The larger the batch size, the longer the time needed for mixing to be sure that the ingredients are evenly distributed throughout the filled vessel. The amount of time required for mixing will be ascertainable by one of ordinary skill in the art.

The filled vessel may be stored away from light and in a dark storage place that is heat and light controlled to ensure that the decarboxylation process does not occur, and so that light does not permeate and compromise the raw materials and carrier. The controlled environment may have a temperature of about 50° F. to about 75° F. The controlled environment may be at standard atmospheric pressure, or about 14.7 lbs per sq inch. The period of time for storage may be about 4 to about 14 weeks, about 4 to about 10 weeks, or about 4 to about 8 weeks. The period of time for storage may be about 4 weeks, about 5 weeks, about 6 weeks, about 7 weeks, or about 8 weeks. The length of storage time may depend on the desired end product. When the goal is to obtain higher concentrations of CBDa or CBG, a longer storage time may be applied, so that the greater concentration of CBDa and CBG, as applicable, is extracted into the carrier. When the goal is to obtain higher concentrations of CBDv, shorter storage periods may be applied.

During storage, over the period of time, substantially all of the cannabinoids and terpenes present in the hemp are extracted from the raw material into the carrier to make the tincture. With cold extraction, the delicate cannabinoids and terpenes are maintained without destroying the chemical structures and transferred into the carrier. During the cold extraction, the temperature may be maintained throughout the process at a temperature below about 75° F. to ensure that the decarboxylation process of the cannabinoids does not occur.

The light-blocking vessel may be of any shape or size as readily understood in the art. The light-blocking vessel may be made from glass, steel, titanium, or any other suitable material.

After storage, the contents within the filled vessel are drained and strained to remove any fiber or other solids resulting in the tincture which, after cold extraction, includes the carrier and the extracted cannabinoids and terpenes. The tincture may be processed for encapsulation (pressed/pill format, enclosed in a gel and or empty capsules), or bottled, e.g., in a light blocking container. It may also be formulated as an oral melt, trope, spray, or vaginal and rectal suppository, or for transdermal application, or aerosol or nasal inhalation. The final product may be kept away from direct sunlight to avoid degradation.

Cold extraction processing as used herein is not a mechanical extraction process involving any type of hydraulic press or otherwise, such as used in cold press extraction. Cold extraction processing is achieved by following the storage and temperature parameters provided herein. Cold extraction processing is achieved without use of a heat source.

The tincture may be administered orally to a user, e.g., human or animal, as a cold or room temperature oil tincture, and/or it may be administered orally after applying heat, i.e., warming, the tincture. The tincture may be warmed by the use of any heat source, such as a stove or in the microwave, to convert the CBDa into CBD.

Administration of the tincture is possible in its cold form or warmed and decarboxylated to provide different benefits the user, for example, based on what works best for their specific body and what processes best for their endocannabic delivery system. Each user is different and metabolizes compounds in a unique way based on a number of factors/variables (i.e., weight, metabolic factors, endocannabic deficiency's, surgeries, malabsorption etc). The inventive tincture allows the user to decide how best to consume the product based upon their specific biology and bioavailability of how they break down these compounds. The user and/or administrator has control over consumption based upon how the product works best for that user.

Also disclosed is a method of reducing or treating arthritic pain or muscle pain by administering a tincture disclosed herein. The tincture may be administered topically, rectally, vaginally, orally, or by inhalation. The tincture may be administered at room temperature, cooled or warmed. Boiling of the tincture may be avoided though it may be added to a hot food or beverage item. The dosing regimen may vary greatly between each user and may be modified to suit the user and the need. For example, about 1 ml to about 20 ml, about 2 ml to about 12 ml, about 5 ml to about 10 ml, or about 5 ml to about 7.5 ml may be administered one to four times daily. About 1 ml to about 20 ml, about 2 ml to about 12 ml, about 5 ml to about 10 ml, or about 5 ml to about 7.5 ml may be administered twice daily.

The dosing regimen may change over time. For example, the user may begin with micro-dosing of the tincture and increase the amount over some period of time.

A method of reducing or treating arthritic pain or muscle pain, insomnia or inflammation may comprise administering about 1 ml about every two hours until pain subsides. A method of reducing or treating arthritic pain or muscle pain, insomnia or inflammation may comprise administering about 1 ml for a period of time, followed by increasing the dosage amount to about 2 ml to about 10 ml one to four times per day.

Also disclosed is a method of preventing or treating a coronavirus by administering the tincture. The tincture may be administered topically, rectally, vaginally, orally, or by inhalation. To prevent or treat coronavirus, the tincture may be administered orally in about 1 ml to about 20 ml, about 2 ml to about 12 ml, about 5 ml to about 10 ml, or about 5 ml to about 7.5 ml one to four times daily. To prevent or treat coronavirus, the tincture may be administered orally in about 1 ml to about 20 ml, about 2 ml to about 12 ml, about 5 ml to about 10 ml, or about 5 ml to about 7.5 ml twice daily.

The dosing regimen for the warmed tincture may be the same or different than the dosing regimen for the cold/unwarmed tincture

Other methods include administering the tincture to treat or reduce anxiety, treat or reduce inflammation, slow tumor growth, reduce symptoms of nausea and vomiting, stimulate appetite, and reduce seizure activity. Other methods include administering the tincture to treat grey matter heterotopia, an autoimmune disease, such as Crohn's and celiac disease, GERD, insomnia, parkinson's, and/or bursitis. The dosing regimens for these methods may be the same as the dosing regimens described above.

### EXAMPLES

### Example 1

A tincture having the following composition was made according to the following processing steps. Dried chardonnay and CBG flower were selected and processed. The Chardonnay and CBG flower were placed in a light-blocking vessel and enough olive oil was added to cover all of the hemp flowers. The hemp flowers and olive oil were mixed to evenly distribute the ingredients, and then stored for about 7 to 8 weeks, at approximately 67 degree F. The contents were mixed about once a week. At the end of the storage period, the carrier/olive oil was strained and separated from the solids and fibers of the hemp flower to result in the tincture.

The tincture prepared according to the process described above was 0.83 wt. % total cannabinoids. The cannabinoid content is shown in Table 1.

TABLE 1

| | Mg per 1 oz. serving | | percent by mass |
|---|---|---|---|
| Total Cannabinoids | 234.87 | mg | 0.83% |
| CBD | 70.88 | mg | 0.25% |
| CBDa | 51.6 | mg | 0.18% |
| CBDva | 0.9 | mg | 0% |
| CBG | 9.4 | mg | 0.03% |
| CBGa | 92.7 | mg | 0.33% |
| CBC | 5.39 | mg | 0.02% |
| CBCa | 4.0 | mg | 0.01% |

Not detected:Δ9-THC, CBN, CBNa, THCa, Δ8-THC, THCv, THCva, CBDv, CBL, CBLa, CBCv, CBT

### Example 2

A second tincture was prepared in same way as Example 1. The tincture prepared according to the process described above was 0.53 wt. % total cannabinoids, and contained 0.22 wt. % terpenes. The cannabinoid content is shown in Table 2. Terpene profile is in Table 3.

TABLE 2

| | mg per 1 gram serving | | percent by mass |
|---|---|---|---|
| Total Cannabinoids | 6.29 | mg | 0.63% |
| CBD | 1.4 | mg | 0.14% |
| CBDa | 2.41 | mg | 0.24% |
| CBG | 0.1 | mg | 0.01% |
| CBGa | 2.05 | mg | 0.21% |
| CBC | 0.13 | mg | 0.01% |
| CBCa | 0.2 | mg | 0.02% |

Not detected:Δ9-THC CBN, THCa, CBDva, Δ8-THC, THCv, THCva, CBDv, CBL, CBLa, CBCv, CBT

TABLE 3

| Terpene | |
|---|---|
| beta-Myrcene | 0.02% |
| Linalool | 0.02% |
| Isopulegol | 0.02% |
| beta-Caryophyllene | 0.02% |
| Guaiol | 0.02% |
| Caryophyllene Oxide | 0.02% |
| alpha-Pinene | 0.01% |
| Camphene | 0.01% |
| beta-Pinene | 0.01% |
| delta-3-Carene | 0.01% |
| alpha-Ocimene | 0.01% |
| D-Limonene | 0.01% |
| gamma-Terpinene | 0.01% |
| alpha-Humulene | 0.01% |
| cis-Nerolidol | 0.01% |
| trans-Nerolidol | 0.01% |

Not detected: alpha-Terpinene, para-Cymene, beta-Ocimene, Eucalyptol, Terpinolene, Geraniol, alpha-Bisabolol

### Example 3

A third tincture was prepared in same way as Example 1, except that MCT oil was used in place of olive oil. The tincture prepared according to the process described above was 0.67 wt. % total cannabinoids, and contained 0.15 wt. % terpenes. The cannabinoid content is shown in Table 4. Terpene profile is in Table 5.

### Table 4

mg per 1 gram serving percent by mass
Total Cannabinoids 6.69 mg 0.67%
THC 0.08 mg 0.01%
CBD 2.25 mg 0.23%
mg per 1 gram serving percent by mass
CBDa 3.17 mg 0.32%
CBGa 0.85 mg 0.09%
CBC 0.14 mg 0.01%
CBCa 0.2 mg 0.02%
Not detected: CBN, THCa, CBDva, Δ8-THC, CBG, THCv, THCva, CBDv, CBL, CBLa, CBCv, CBT

TABLE 5

| Terpene | |
|---|---|
| alpha-Pinene | 0.02% |
| Camphene | 0.02% |
| beta-Myrcene | 0.02% |
| beta-Pinene | 0.02% |
| delta-3-Carene | 0.02% |
| beta-Caryophyllene | 0.02% |
| alpha-Humulene | 0.02% |
| alpha-Ocimene | 0.01% |

Not detected: alpha-Terpinene, D-Limonene, para-Cymene, beta-Ocimene, Eucalyptol, gamma-Terpinene, Terpinolene, Linalool, Isopulegol, Geraniol, cis-Nerolidol, trans-Nerolidol, Guaiol, Caryophyllene Oxide, alpha-Bisabolol

While there have been described what are presently believed to be various aspects and certain desirable embodiments of the disclosure, those skilled in the art will recognize that changes and modifications may be made thereto without departing from the spirit of the disclosure, and it is intended to include all such changes and modifications as fall within the true scope of the disclosure.

What is claimed:

1. A tincture containing cannabinoids comprising:
a) cannabinoids extracted from hemp, wherein the cannabinoids are selected from the group consisting of: CBD, CBDa, CBDva, CBG, CBGa, THC, THCa, THCv, CBGv, CBGva, CBDv, CBCv, CBC, CBCa, CBGM, CBN, CBL, CBLa, and any combination thereof;
b) terpenes extracted from the hemp, wherein the terpenes are selected from the group consisting of: Myrcene, Beta-caryophyllene, Limonene, Linalool, Pinene, Humulene, Terpinolene, Alpha-bisabolol, Eucalyptol, Geraniol, Terpineol, Farnesene, Borneol, Ocimene, Nerolidol, Guaiol, Valencene, Delta-3-carene, Phytol, Sabinene, Phellandrene, Menthol, Fenchol, Terpinene, Isoborneol, Cymene, Octanol, Cedrene, Camphene, Geranyl Acetate, Bergamotene, Camphor, Pulegon, and any combination thereof; and
b) a carrier;
wherein substantially all of the cannabinoids and terpenes from the hemp are present in the tincture.

2. The tincture of claim 1, wherein the carrier is a carrier oil selected from the group consisting of: Olive oil, Hempseed oil, Fractionated MCT oil, Coconut oil, Avocado oil, Almond oil, Cod Liver oil, Sunflower oil, Peanut oil, any known vegetable oil, or any combination thereof.

3. The tincture of claim 1, wherein the cannabinoids are present in about 0.4 wt. % to about 1.5 wt. %, and the terpenes are present in about 0.1 wt. % to about 0.3 wt. %, by total weight of the tincture.

4. The tincture of claim 1, wherein the cannabinoids comprise CBD, CBDa, CBGa, CBC, CBCa, and at least one additional cannabinoid.

5. The tincture of claim 4, wherein the at least one additional cannabinoid is selected from the group consisting of: THC, CBDva, CBG, CBNa, and any combination thereof.

6. The tincture of claim 1, wherein the cannabinoids comprise: CBD in about 0.1 wt % to about 0.3 wt %; CBDa in about 0.1 wt % to about 0.3 wt %; CBGa in about 0.1 wt % to about 0.35 wt %; CBC in about 0.01 wt % to about 0.02 wt %; and CBCa in about 0.01 wt % to about 0.02 wt %, by total weight of the tincture.

7. The tincture of claim 1, wherein the terpenes comprise Beta-Myrcene, Beta-caryophyllene, Alpha-Pinene, Beta-Pinene, Camphene, Delta-3-carene, Alpha-Humulene, and Alpha-Ocimene.

8. The tincture of claim 1, wherein the tincture comprises about 0.01 wt. % to about 0.02 wt. % of Beta-Myrcene, about 0.01 wt. % to about 0.02 wt. % of Beta-Caryophyllene, about 0.01 wt. % to about 0.02 wt. % of Alpha-Pinene, about 0.01 wt. % to about 0.02 wt. % of Beta-Pinene, about 0.01 wt. % to about 0.02 wt. % of Camphene, about 0.01 wt. % to about 0.02 wt. % of Delta-3-carene, and about 0.01 wt. % to about 0.02 wt. % of Alpha-Humulene.

9. A method of preventing or treating a coronavirus by administering the tincture of claim 1.

10. The method of claim 9, wherein about 1 ml to about 20 ml of the tincture is administered orally one to four times daily.

11. A method of reducing or treating arthritic pain or muscle pain, reducing or treating anxiety, reducing or treating inflammation, slowing tumor growth, reducing or treating symptoms of nausea and vomiting, stimulating appetite, and/or reducing seizure activity, by administering the tincture of claim 1.

12. The method of claim 11, wherein the tincture is administered topically, orally, vaginally, rectally, or by inhalation.

13. The method of claim 11, wherein about 1 ml to about 20 ml of the tincture is administered orally one to four times daily.

14. A method of making a tincture of claim 1 by cold extraction, the method comprising the steps of:
i) supplying raw hemp;
ii) placing the hemp in a light-blocking vessel;
iii) adding a carrier to the vessel make a filled vessel;
iv) mixing the hemp and carrier in the filled vessel to evenly distribute the ingredients;
v) storing the filled vessel in a controlled environment for a period of time; and
vi) mixing the filled vessel at least one time per week throughout the period of time.

15. The method of claim 14, wherein the period of time of storage is about 4 weeks to about 8 weeks.

16. The method of claim 14, wherein the controlled environment is at a temperature of about 50° F. to about 75° F. and at about standard atmospheric pressure.

17. The method of claim 14, wherein the carrier is mixed with at least one additive before step (iii).

18. The method of claim 14, wherein step v) further includes: extracting cannabinoids and terpenes from the hemp into the carrier.

19. The method of claim 14, wherein the method of cold extraction is maintained at a temperature below about 75° F.

20. The tincture of claim 1, produced by cold extraction processing.
